(19) United States
(12) Patent Application Publication
(10) Pub. No.: US 2026/0001024 A1
(43) Pub. Date: Jan. 1, 2026

(54) Dehumidifying Bag with Inner Cavity

(71) Applicant: GUANGDONG ILOVENUMBERONE CO.,LTD, Guangzhou (CN)

(72) Inventor: Changhui LIU, Guangzhou (CN)

(21) Appl. No.: 18/760,030

(22) Filed: Jul. 1, 2024

(51) Int. Cl. B01D 53/04; A61L 9/12; B01D 53/26

(52) U.S. Cl. CPC B01D 53/0407; A61L 9/12; B01D 53/261; A61L 2209/15; A61L 2209/22; B01D 2257/80; B01D 2257/90

(57) ABSTRACT

The present utility model discloses a dehumidifying bag with an inner cavity, being provided with an inner cavity body and a breathable bag. The inner cavity body consists of a storage port, a carrier and a plurality of partitions. The plurality of partitions are provided in the carrier, so that the carrier forms a plurality of independent chambers; a part of an inner wall of the breathable bag is closely connected to a circumference of a side wall of the storage port, and a surface of the breathable bag is provided with a breathable film; and a moisture-absorbing material or an aromatic or deodorizing or purifying or insecticidal material is placed in the plurality of independent chambers. The present utility model is simple in structure and convenient to use, and can simultaneously achieve the functions of moisture absorption, aromatizing, deodorizing, purifying and disinsection.

### TECHNICAL FIELD

The present utility model relates to the technical field of dehumidifying, and in particular, to a dehumidifying bag with an inner cavity.

### BACKGROUND

As one of the daily necessities, dehumidifying boxes are used to absorb the moisture in the humid air in humid areas, and thus are used by many families. A current dehumidifying box has a single function. Even for some dehumidifying boxes have dehumidifying materials, aromatic materials, deodorizing or insecticidal materials mixed together, the functions are impaired, which compromises the original role of the dehumidifying box. In addition, for an existing bagged moisture-absorbing product, an upper moisture-absorbing area and a lower liquid storage area are separated by heat sealing, which easily results in that the liquid fail to flow into the lower liquid storage area normally after moisture absorption. Moreover, as a water gap between the upper moisture-absorbing area and the lower liquid storage area becomes extremely small after the heat sealing, it will hinder the liquid storage area from further moisture absorption. Therefore, a maximum moisture-absorbing amount of the dehumidifying material cannot be realized, and thereby cause waste.

### SUMMARY

In view of the shortcomings of the existing technology, the present utility model aims to provide a dehumidifying bag with an inner cavity. Through structures of an inner cavity body and a breathable bag, the defects of an existing dehumidifying bag can be overcome, and a variety of different materials may be placed in the inner cavity body at the same time, so that one product may have multiple different functions.

In order to achieve the above purposes, the present utility model adopts the following technical solution:

A dehumidifying bag with an inner cavity, being provided with an inner cavity body, and a breathable bag enclosing the inner cavity body, the inner cavity body consisting of a storage port, a carrier and a plurality of partitions, where the plurality of partitions are provided in the carrier, so that the carrier forms a plurality of independent chambers; a part of an inner wall of the breathable bag is closely connected to a circumference of a side wall of the storage port, and a surface of the breathable bag is provided with a breathable film; and a moisture-absorbing material or an aromatic or deodorizing or purifying or insecticidal material is placed in the plurality of independent chambers.

It should be noted that a surface of the inner cavity body is provided with a plurality of diversion trenches or diversion holes.

It should be noted that a bottom of the inner cavity body is provided with a plurality of diversion trenches or diversion holes.

It should be noted that a gap is provided between the inner cavity body and the breathable bag.

It should be noted that the partition is provided along a length direction of the carrier.

It should be noted that the storage port, the carrier and the plurality of partitions are of an integrally-formed structure.

It should be noted that the breathable film may be equivalent to a surface area of the breathable bag, or the breathable film occupies a part of the surface area of the breathable bag.

It should be noted that a top of the breathable bag is provided with an opening corresponding to the position of the storage port.

It should be noted that a hanger is further included, and the hanger is connected to the breathable bag.

It should be pointed out that except for the opening, the remaining circumference of the breathable bag of the present utility model may be bonded by heat pressing, or made by other prior technologies.

It should be further noted that the hanger of the present utility model may use a soft material, and use a simple hook. For example, a perforation is formed in the top of the breathable bag, and the hanger penetrates into and is connected to the perforation. In addition, the hanger may be made of silica gel. In this way, it can ensure that the hanger will not be damaged even it is squeezed in the process of packaging or transportation, and it can save space during packaging.

In addition, as a deformation, there may be no hanger. For example, a consumer may directly use a self-made rope to replace the hanger.

It should be pointed out that the opening used for hanging may be in any geometric shape.

As a preferred technical solution, the opening of the breathable bag of the present utility model may be sealed by a pulling strip.

It should be pointed out that the inner cavity body in the present utility model may be in any existing geometric shape.

The beneficial effects of the present utility model lie in: it is simple in structure and convenient to use; it can simultaneously achieve the functions of moisture absorption, atomization, deodorizing, purifying and disinsection; secondly, due to an “inside and outside” structure is used, the defects of the existing dehumidifying bag have been completely solved, ensuring that the moisture after moisture absorption can completely drip into the breathable bag, giving a full play to the moisture-absorbing function of the moisture-absorbing material, and also ensuring that all functions do not interfere with each other; thirdly, by changing the form of the hanger, the dehumidifying bag can be hung in different environments; and finally, by opening the dehumidifying bag, materials with different functions may be added and the moisture contained in the breathable bag may also be poured out to achieve recycling.

### BRIEF DESCRIPTION OF FIGURES

FIG. 1 is a schematic structural diagram of the present utility model.

FIG. 2 is a schematic structural diagram of an inner cavity body in FIG. 1 from another angle.

### DETAILED DESCRIPTION

The present utility model will be further described below in combination with the drawings. It should be noted that the present embodiment takes the present technical solution as a premise and gives detailed implementations and specific operation process, but the protection scope of the present utility model is not limited to the present embodiment.

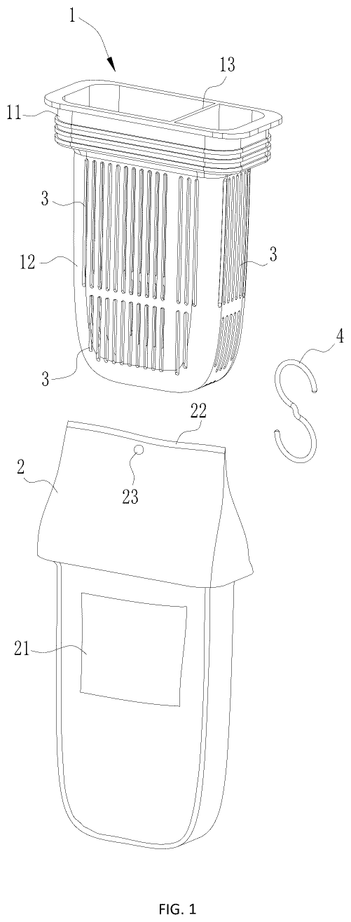

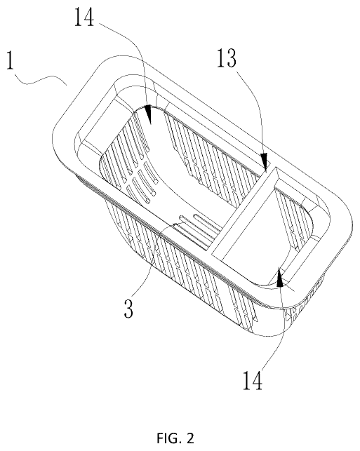

As shown in FIG. 1 and FIG. 2, the present utility model is a dehumidifying bag with an inner cavity. The dehumidifying bag is provided with an inner cavity body 1, and a breathable bag 2 enclosing the inner cavity body 1. The inner cavity body 1 consists of a storage port 11, a carrier 12 and a plurality of partitions 13. The plurality of partitions 13 are provided in the carrier 12, so that the carrier 12 forms a plurality of independent chambers 14. A part of an inner wall of the breathable bag 2 is closely connected to a circumference of a side wall of the storage port 11. A surface of the breathable bag 2 is provided with a breathable film 21. A moisture-absorbing material or an aromatic or deodorizing or purifying or insecticidal material is placed in the plurality of independent chambers 14.

It should be pointed out that in order to prevent the moisture-absorbing material or the aromatic or deodorizing or purifying or insecticidal material from accidentally falling into the breathable bag, a top surface of the inner cavity body may be sealed with an airtight plastic film.

It should be further noted that the moisture-absorbing material contained in the inner cavity body absorbs the moisture of the humid air in the environment, which drips onto a bottom of the breathable bag, and thus a lower part of the breathable bag serves as a liquid storage area.

It should be pointed out that the connection between the inner cavity body and the breathable bag mentioned in the present utility model may be heat pressing in the prior art, but the connection is not limited to heat pressing. That is, the modes through which the inner cavity body can be tightly connected to the breathable bag should also be deformations of the present utility model.

Further, as shown in FIG. 1 and FIG. 2, a surface of the inner cavity body 1 of the present utility model is provided with a plurality of diversion trenches 3 or diversion holes.

Further, as shown in FIG. 2, a bottom of the inner cavity body 1 of the present utility model is provided with a plurality of diversion trenches 3 or diversion holes.

Further, a gap is provided between the inner cavity body and the breathable bag of the present utility model. Moreover, that is, the inner cavity body and the breathable bag are partially connected to each other, and the remaining parts are not connected. In this way, it can fully ensure that the moisture absorbed by the moisture-absorbing material can completely drip into the breathable bag, giving a full play to the performance of the moisture-absorbing material.

Further, the partition of the present utility model is provided along a length direction of the carrier.

Further, the storage port, the carrier and the plurality of partitions of the present utility model are of an integrally-formed structure.

Further, as shown in FIG. 1, the breathable film 21 of the present utility model may be equivalent to a surface area of the breathable bag, or the breathable film 21 occupies a part of the surface area of the breathable bag. It should be noted that the breathable film with different areas will not affect the performance of the moisture-absorbing material in principle, but only affect the speed of moisture absorption.

Further, as shown in FIG. 1, a top of the breathable bag 2 of the present utility model is provided with an opening 22 corresponding to the position of the storage port 11. The moisture-absorbing material or the aromatic or deodorizing or purifying or insecticidal material is placed in the inner cavity body through the opening. Moreover, when enough moisture is collected in the breathable bag, the moisture is poured out through the opening.

Further, as shown in FIG. 1, the present utility model further includes a hanger 4. The hanger 4 is connected to the breathable bag 2.

It should be pointed out that except for the opening, the remaining circumference of the breathable bag of the present utility model may be bonded by heat pressing, or made by other prior technologies.

It should be further noted that the hanger of the present utility model may use a soft material, and use a simple hook. For example, as shown in FIG. 1, a perforation 23 is formed in the top of the breathable bag 1. The hanger 4 penetrates into and connected to the perforation 23. In addition, the hanger may be made of silica gel. In this way, it can ensure that the hanger will not be damaged even it is squeezed in the process of packaging or transportation, and it can save space during packaging.

In addition, as a deformation, there may be no hanger. For example, a consumer may directly use a self-made rope to replace the hanger.

It should be pointed out that the opening used for hanging may be in any geometric shape.

As a preferred technical solution, the opening of the breathable bag of the present utility model may be sealed by a pulling strip.

It should be pointed out that the inner cavity body in the present utility model may be in any existing geometric shape.

It should be pointed out that a shape of the breathable bag of the present utility model may be similar to that of the inner cavity body, or both are different shapes.

It should be noted that a shape of the diversion trench or the diversion hole of the present utility model is not limited and may be made according to the actual needs.

In actual use, the present utility model may be packed with a packaging bag, and used by opening an outer packaging; or, an airtight film is adhered to the surface of the breathable film of the breathable bag, and the present utility model is used directly after tearing off the airtight film.

### Embodiment

A breathable bag is opened, then different materials are placed in a plurality of independent cavity bodies in an inner cavity body, and then the breathable bag is resealed. A user can use it normally after hanging it with a hanger or a self-made hanging rope. A moisture-absorbing material in the inner cavity body will absorb the moisture in the air through a breathable film of the breathable bag, and then the excess moisture will drip into the breathable bag from a diversion trench and a diversion hole formed in the inner cavity body; and other materials in the inner cavity body, such as an aromatic or deodorant or purifying agent or insecticide are also distributed through the breathable film on the breathable bag.

For those skilled in the art, various corresponding changes and deformations may be given according to the above technical solution and concept, and all these changes and deformations should be included in the protection scope of the claims of the present utility model.

What is claimed is:

1. A dehumidifying bag with an inner cavity, being provided with an inner cavity body, and a breathable bag enclosing the inner cavity body, the inner cavity body consisting of a storage port, a carrier and a plurality of partitions, wherein the plurality of partitions are provided in the carrier, so that the carrier forms a plurality of independent chambers; a part of an inner wall of the breathable bag is closely connected to a circumference of a side wall of the storage port, and a surface of the breathable bag is provided with a breathable film; and a moisture-absorbing material or an aromatic or deodorizing or purifying or insecticidal material is placed in the plurality of independent chambers.

2. The dehumidifying bag with an inner cavity according to claim 1, wherein a surface of the inner cavity body is provided with a plurality of diversion trenches or diversion holes.

3. The dehumidifying bag with an inner cavity according to claim 1 or 2, wherein a bottom of the inner cavity body is provided with a plurality of diversion trenches or diversion holes.

4. The dehumidifying bag with an inner cavity according to claim 1, wherein a gap is provided between the inner cavity body and the breathable bag.

5. The dehumidifying bag with an inner cavity according to claim 1, wherein the partition is provided along a length direction of the carrier.

6. The dehumidifying bag with an inner cavity according to claim 1, wherein the storage port, the carrier and the plurality of partitions are of an integrally-formed structure.

7. The dehumidifying bag with an inner cavity according to claim 1, wherein the breathable film may be equivalent to a surface area of the breathable bag, or the breathable film occupies a part of the surface area of the breathable bag.

8. The dehumidifying bag with an inner cavity according to claim 1, wherein a top of the breathable bag is provided with an opening corresponding to the position of the storage port.

9. The dehumidifying bag with an inner cavity according to claim 1, further comprising a hanger, and the hanger is connected to the breathable bag.
